(19) United States
(12) Patent Application Publication
(10) Pub. No.: US 2026/0263102 A1
(43) Pub. Date: Sep. 10, 2026

(54) SURGICAL END EFFECTOR ASSEMBLIES SUCH AS FOR USE IN SURGICAL ROBOTIC SYSTEMS AND METHODS OF MANUFACTURING THE SAME

(71) Applicant: Covidien LP

(72) Inventors: Allen E. Honegger, Thornton, CO (US); James L. Morris, III, Denver, CO (US); Curtis M. Siebenaller, Frederick, CO (US); Ryan C. Fahrenkrug, Loveland, CO (US); Carolyn R. Girvin, Boulder, CO (US); Dylan R. Kingsley, Broomfield, CO (US); Christopher L. Tschudy, Arvada, CO (US); Jason G. Weihe, Longmont, CO (US); William R. Whitney, Boulder, CO (US); Matthew D. Straka, Arvada, CO (US)

(21) Appl. No.: 19/166,424

(22) PCT Filed: Mar. 11, 2024
(86) PCT No.: PCT/IB2024/052347

§ 371 Date: Sep. 17, 2025

Related U.S. Application Data
(60) Provisional application No. 63/453,771, filed on Mar. 22, 2023.

(51) Int. Cl. A61B 17/29; A61B 17/00; A61B 34/30

(52) U.S. Cl. CPC A61B 17/29; A61B 34/30; A61B 2017/00526; A61B 2017/294

(57) ABSTRACT

A method of assembling a surgical end effector includes positioning a first proximal flag (143a) of a first jaw member (142) second and third proximal flags (145a) a second jaw member (144) and adjacent to the second flag such that cam slots of the first and second flags are partially aligned and define a first passage, inserting a cam head assembly between the first and third flags in a first orientation wherein a first cam pin portion (818a) extending from a cam block of the cam head assembly is oriented substantially parallel relative to the flags, and rotating the cam head assembly from the first orientation to a second orientation such that the first cam pin portion is oriented substantially perpendicularly relative to the flags and whereby the first cam pin portion rotates through the first passage into engagement within the cam slots.

### CROSS-REFERENCE TO RELATED APPLICATIONS

This application claims the benefit of U.S. Provisional Patent Application Ser. No. 63/453,771, filed Mar. 22, 2023, the entire content of which is incorporated herein by reference.

### FIELD

This disclosure relates to surgical instruments and, more particularly, to surgical end effector assemblies such as for use in surgical robotic systems and methods of manufacturing such surgical end effector assemblies.

### BACKGROUND

Robotic surgical systems are increasingly utilized in various different surgical procedures. Some robotic surgical systems include a console supporting a robotic arm. One or more different surgical instruments may be configured for use with the robotic surgical system and selectively mountable to the robotic arm. The robotic arm provides one or more inputs to the mounted surgical instrument to enable operation of the mounted surgical instrument, e.g., to rotate, articulate, and/or actuate the mounted surgical instrument.

End effector assemblies suitable for use with the surgical instruments of robotic surgical systems, or any other surgical instruments, may include jaw-based end effector assemblies. A jaw-based end effector assembly typically includes one or more jaw members movable to grasp tissue between the jaw member and an opposing structure, e.g., another jaw member. In order to actuate the one or more jaw members, various different actuation mechanisms may be employed such as, for example, a cam mechanism involving movement of a camming structure through cam slots to thereby move the one or more jaw members.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any of the aspects described herein may be used in conjunction with any or all of the other aspects described herein.

Provided in accordance with aspects of this disclosure is a method of assembling an end effector assembly of a surgical instrument. The method includes positioning a first proximal flag of a first jaw member between spaced apart second and third proximal flags of a second jaw member and adjacent to the second proximal flag such that a first cam slot defined through the first proximal flag is partially aligned with a second cam slot defined through the second proximal flag to define a first passage through the first and second cam slots. The method further includes inserting a cam head assembly between the first and third proximal flags in a first orientation wherein a first cam pin portion extending from a cam block of the cam head assembly is oriented substantially parallel relative to the first, second, and third proximal flags. The method also includes rotating the cam head assembly from the first orientation to a second orientation, wherein the first cam pin portion is oriented substantially perpendicularly relative to the first, second, and third proximal flags. This rotation rotates the first cam pin portion through the first passage and into engagement within the first and second cam slots.

In an aspect of this disclosure, the first jaw member further includes a fourth proximal flag spaced apart from the first proximal flag. In such aspects, the positioning further includes positioning the fourth proximal flag adjacent to the third proximal flag such that a fourth cam slot defined through the fourth proximal flag is partially aligned with a third cam slot defined through the third proximal flag to define a second passage through the third and fourth cam slots, the cam head assembly includes a second cam pin portion extending from the cam block opposite the first cam pin portion, and the rotating rotates the second cam pin portion through the second passage and into engagement within the third and fourth cam slots.

In another aspect of this disclosure, positioning the fourth proximal flag adjacent to the third proximal flag includes positioning the fourth proximal flag on an interior side of the third proximal flag between the third proximal flag and the first proximal flag. Alternatively, positioning the fourth proximal flag adjacent to the third proximal flag includes positioning the fourth proximal flag on an exterior side of the third proximal flag such that the third proximal flag is disposed between the fourth proximal flag and the first proximal flag.

In still another aspect of this disclosure, the cam block includes a first corner defining a chamfer, and the rotating includes rotating the first corner relative to the first proximal flag with a clearance therebetween defined by the chamfer.

In yet another aspect of this disclosure, the first jaw member includes a first jaw body extending distally from the first proximal flag and defining a first tissue contacting surface, the second jaw member includes a second jaw body extending distally from the second and third proximal flags and defining a second tissue contacting surface, and the positioning includes positioning the first and second tissue contacting surfaces relative to one another at an angle of from about 150 degrees to about 175 degrees.

In still yet another aspect of this disclosure, after the rotating, the method further includes positioning the first proximal flag relative to the second and third proximal flags such that a first pivot aperture defined through the first proximal flag is aligned with second and third pivot apertures defined through the second and third proximal flags. In such aspects, the method further includes inserting a pivot pin through the aligned first, second, and third pivot apertures to thereby pivotably couple the first and second jaw members with one another.

In another aspect of this disclosure, the method further includes retaining the pivot pin in engagement within the first, second, and third pivot apertures to thereby retain the first and second jaw members in pivotable engagement with one another.

In another aspect of this disclosure, the cam head assembly includes a drive tube engaged with and extending proximally from the cam block prior to the inserting.

In yet another aspect of this disclosure, the method also includes welding the first cam pin portion to the cam block prior to the inserting.

In still another aspect of this disclosure, the positioning includes inserting the first and second jaw members into a fixture, and wherein the inserting and the rotating are performed with the first and second jaw members disposed within the fixture.

Another method of assembling an end effector assembly of a surgical instrument provided in accordance with this disclosure includes positioning first and fourth proximal flags of a first jaw member relative to second and third proximal flags of a second jaw member such that the first and second proximal flags are adjacent to one another and define a first passage through respective first and second cam slots of the first and second proximal flags, and such that the third and fourth proximal flags are adjacent to one another and define a second passage through respective third and fourth cam slots of the third and fourth proximal flags. In such a configuration, the inner-most two proximal flags of the first, second, third, and fourth proximal flags define a distance therebetween. The method further includes inserting a cam head assembly between the inner-most two proximal flags, the cam head assembly including a cam block and cam pin engaged with the cam block such that first and second cam pin portions extend from opposing sides of the cam block. The cam pin defines a length greater than the distance between the inner-most two proximal flags. The method also includes rotating the cam head assembly from a first orientation to a second orientation to thereby rotate the cam pin such that the first and second cam pin portions are moved through the respective first and second passages with the first cam pin portion engaged within the first and second cam slots and the second cam pin portion engaged within the third and fourth cam slots, thereby engaging the first and second jaw members with the cam head assembly.

In an aspect of this disclosure, the cam block defines a first diagonal dimension greater than the distance between the inner-most two proximal flags and a second, opposing diagonal dimension less than the distance between the inner-most two proximal flags to define a one-way direction of rotation for the rotating.

In another aspect of this disclosure, the method further includes, prior to the positioning, welding the cam pin within the cam block.

In still another aspect of this disclosure, the positioning includes inserting the first and second jaw members into a fixture, and wherein the inserting and the rotating are performed with the first and second jaw members disposed within the fixture.

In yet another aspect of this disclosure, the method includes, after the rotating, positioning the first and second jaw members such that pivot apertures defined through the first and second jaw members are aligned with one another, and inserting a pivot pin through the aligned pivot apertures to thereby pivotably couple the first and second jaw members with one another.

An end effector assembly of a surgical instrument provided in accordance with this disclosure includes a first jaw member, a second jaw member, a pivot, and a cam drive assembly. The first jaw member includes a first pair of proximal flags and a distal body extending distally from the first pair of proximal flags and defining a first tissue contacting surface. The proximal flags of the first pair of proximal flags define a transverse distance therebetween. The second jaw member includes a second pair of proximal flags and a distal body extending distally from the second pair of proximal flags and defining a second tissue contacting surface. The first pair of proximal flags is disposed between the proximal flags of the second pair of proximal flags. The pivot pivotably couples the first and second pairs of proximal flags with one another to enable pivoting of at least one of the first or second tissue contacting surfaces relative to another of the first or second tissue contacting surfaces between spaced apart and approximated positions for grasping tissue therebetween. The cam drive assembly includes a cam block and a cam pin fixed relative to the cam block. The cam block is disposed between the proximal flags of the first pair of proximal flags with the cam pin operably engaging cam slots defined within the proximal flags of the first and second pairs of proximal flags. The cam block has a rectangular configuration defining a first diagonal transverse dimension and a second diagonal transverse dimension opposite the first diagonal transverse dimension. The first diagonal transverse dimension is greater than the transverse distance and the second diagonal transverse dimension is less than the transverse distance.

In another aspect of this disclosure, the first diagonal transverse dimension extends between first and second corners of the cam block and the second diagonal transverse dimension extends between first and second chamfers defined in the cam block.

In still another aspect of this disclosure, the cam pin extends through the cam block and the cam block defines an aperture extending perpendicularly relative to the cam pin. The cam pin is welded to the cam block within the aperture.

In yet another aspect of this disclosure, the cam drive assembly further includes a proximal extension configured to engage a drive tube with the cam block.

Another end effector assembly of a surgical instrument provided in accordance with this disclosure includes a first jaw member, a second jaw member, a pivot, and a cam drive assembly. The first jaw member includes a pair of first proximal flags and a distal body extending distally from the pair of first proximal flags and defining a first tissue contacting surface. The second jaw member includes at least one second proximal flag and a distal body extending distally from the at least one second proximal flag and defining a second tissue contacting surface. The pivot pivotably couples the pair of first proximal flags and the at least one second proximal flag with one another to enable pivoting of at least one of the first or second tissue contacting surfaces relative to another of the first or second tissue contacting surfaces between spaced apart and approximated positions for grasping tissue therebetween. The cam drive assembly includes a cam block and a cam pin fixed relative to the cam block. The cam block is disposed between the first proximal flags of the pair of first proximal flags with the cam pin operably engaging cam slots defined within the first proximal flags of the pair of first proximal flags. The cam block includes a first diagonal defined between a pair of diagonally opposed chamfered corners.

In an aspect of this disclosure, the first proximal flags of the pair of first proximal flags define a transverse distance therebetween and the first diagonal defines a diagonal transverse distance that is less than the transverse distance.

In another aspect of this disclosure, the cam block further includes a second diagonal opposite the first diagonal and defined between a pair of diagonally opposed unchamfered corners.

In still another aspect of this disclosure, the at least one second proximal flag defines a cam slot. The cam pin, in such aspects, is operably engaged within the cam slot of the at least one second proximal flag.

In yet another aspect of this disclosure, the at least one second proximal flag includes a pair of second proximal flags. In such aspects, the pair of first proximal flags may be disposed between the second proximal flags of the pair of second proximal flags.

The details of one or more aspects of this disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects and features of this disclosure are described hereinbelow with reference to the drawings wherein like numerals designate identical or corresponding elements in each of the several views.

FIG. 1 is a schematic illustration of a surgical robotic system including a control tower, a console, and one or more surgical robotic arms according to aspects of this disclosure;

FIG. 2 is a perspective view of a surgical robotic arm of the surgical robotic system of FIG. 1 according to aspects of this disclosure;

FIG. 3 is a perspective view of a setup arm with the surgical robotic arm of the surgical robotic system of FIG. 1 according to aspects of this disclosure;

FIG. 4 is a schematic diagram of a computer architecture of the surgical robotic system of FIG. 1 according to aspects of this disclosure;

FIG. 5 is a perspective view of a surgical instrument provided in accordance with this disclosure configured for mounting on a robotic arm of a surgical robotic system such as the surgical robotic system of FIG. 1;

FIGS. 6A and 6B are front and rear perspective views, respectively, of a proximal portion of the surgical instrument of FIG. 5, with an outer shell removed;

FIG. 7 is a front perspective view of the proximal portion of the surgical instrument of FIG. 5 with the outer shell and additional internal components removed;

FIGS. 8A and 8B are side and top views, respectively, of the end effector assembly of the surgical instrument of FIG. 5;

FIG. 9A is a perspective view of a cam drive assembly of the surgical instrument of FIG. 5;

FIG. 9B is a rear view of a cam head assembly of the cam drive assembly of FIG. 9A;

FIGS. 10A-10D are progressive illustrations of assembly of the cam drive assembly of FIG. 9A with the jaw members of the end effector assembly of FIGS. 8A and 8B;

FIG. 11 is a flow diagram of a method of assembly in accordance with this disclosure;

FIG. 12 is a top, perspective view of the jaw members of the end effector assembly of FIGS. 8A and 8B positioned in a fixture provided in accordance with this disclosure to facilitate assembly of the cam drive assembly of FIG. 9A with the jaw members;

FIG. 13 is a side view of a portion of the fixture of FIG. 12 with the jaw members of the end effector assembly of FIGS. 8A and 8B positioned therein to facilitate assembly of the cam drive assembly of FIG. 9A with the jaw members; and

FIG. 14 is a top, perspective view of another fixture provided in accordance with this disclosure configured to facilitate assembly of the end effector assembly of FIGS. 8A and 8B.

### DETAILED DESCRIPTION

This disclosure provides surgical end effector assemblies and methods of manufacturing such surgical end effector assemblies. As described in detail below, the surgical end effector assemblies of this disclosure may be configured for use with a surgical robotic system, which may include, for example, a surgical console, a control tower, and one or more movable carts having a surgical robotic arm coupled to a setup arm. The surgical console receives user inputs through one or more interface devices, which are interpreted by the control tower as movement commands for moving the surgical robotic arm. The surgical robotic arm includes a controller, which is configured to process the movement commands and to generate a torque command for activating one or more actuators of the robotic arm, which, in turn, move the robotic arm in response to the movement commands. Although described hereinbelow in connection with surgical robotic systems, the aspects and features of this disclosure may also be adapted for use with handheld surgical instruments such as, for example, endoscopic instruments and/or open instruments.

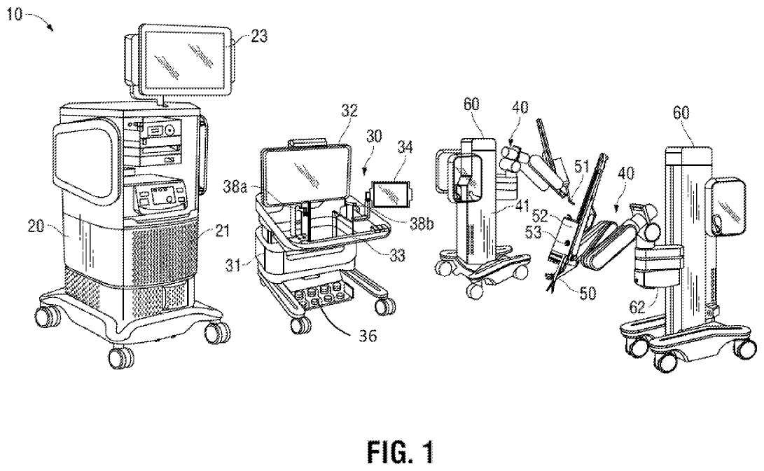

With reference to FIG. 1, a surgical robotic system 10 includes a control tower 20, which is connected to components of the surgical robotic system 10 including a surgical console 30 and one or more robotic arms 40. Each of the robotic arms 40 includes a surgical instrument 50, 51 removably coupled thereto. Each of the robotic arms 40 is also coupled to a movable cart 60.

The one or more surgical instruments 50, 51 may be configured for use during minimally invasive surgical procedures and/or open surgical procedures. In aspects, one of the surgical instruments 51 may be an endoscope, such as an endoscopic camera 51, configured to provide a video feed for the clinician. In aspects, one of the surgical instruments 50 may be a jaw-based surgical instrument such as, for example, an electrosurgical forceps, ultrasonic sealing and dissection instrument, surgical stapling instrument, surgical clip applier, surgical grasper, or any other suitable surgical instrument including an end effector assembly having one or more jaw members. Additionally or alternatively, one of the surgical instruments 50, 51 may include an energizable element (e.g., a monopolar, bipolar, thermal, microwave, etc. element) configured to treat tissue. Suction and/or irrigation functionality for the surgical instruments 50, 51 are also contemplated. Other suitable surgical instruments 50, 51 may also be provided.

Endoscopic camera 51, as noted above, may be configured to capture video of the surgical site. In such aspects, the surgical console 30 includes a first display 32, which displays a video feed of the surgical site provided by endoscopic camera 51, and a second display 34, which displays a user interface for controlling the surgical robotic system 10. The first and second displays 32 and 34 may be touchscreen graphical user interface (GUI) displays allowing for receipt of various user inputs.

The surgical console 30 also includes a plurality of user interface devices, such as foot pedals 36 and a pair of handle controllers 38a and 38b which are used by a clinician to remotely control robotic arms 40. The surgical console further includes an armrest 33 used to support clinician's arms while operating the handle controllers 38a and 38b.

The control tower 20 includes a display 23, which may be a touchscreen GUI, and provides outputs to the various GUIs. The control tower 20 also acts as an interface between the surgical console 30 and one or more robotic arms 40. In particular, the control tower 20 is configured to control the robotic arms 40, such as to move the robotic arms 40 and the corresponding surgical instrument 50, 51, based on a set of programmable instructions and/or input commands from the surgical console 30, in such a way that robotic arms 40 and the surgical instrument 50, 51 execute a desired movement sequence in response to input from the foot pedals 36 and/or the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by this disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth® (an open wireless protocol for exchanging data over short distances, using short length radio waves, from fixed and mobile devices, creating personal area networks (PANs)), and/or ZigBee® (a specification for a suite of high level communication protocols using small, low-power digital radios based on the IEEE 122.15.4-2003 standard for wireless personal area networks (WPANs)).

The computers 21, 31, 41 may include any suitable processor(s) operably connected to a memory, which may include one or more of volatile, non-volatile, magnetic, optical, quantum, and/or electrical media, such as read-only memory (ROM), random access memory (RAM), electrically-erasable programmable ROM (EEPROM), non-volatile RAM (NVRAM), or flash memory. The processor(s) may be any suitable processor(s) (e.g., control circuit(s)) adapted to perform operations, calculations, and/or set of instructions including, but not limited to, a hardware processor, a field programmable gate array (FPGA), a digital signal processor (DSP), a central processing unit (CPU), a microprocessor, a quantum processor, and combinations thereof. Those skilled in the art will appreciate that the processor may be substituted for by using any logic processor (e.g., control circuit) adapted to execute algorithms, calculations, and/or set of instructions.

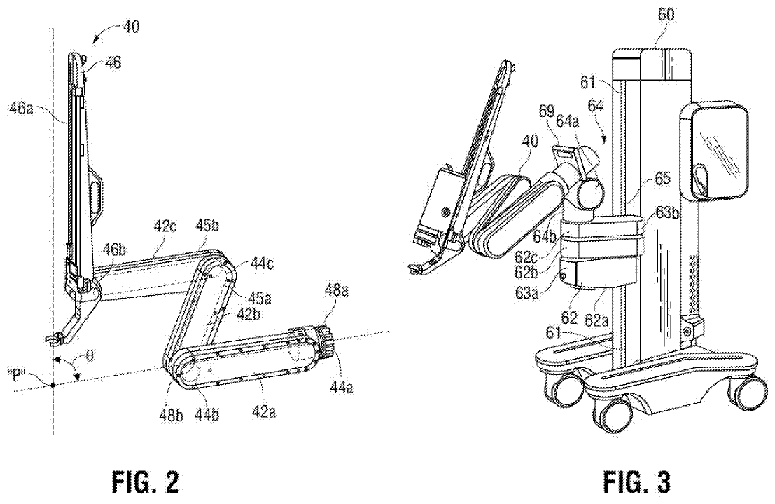

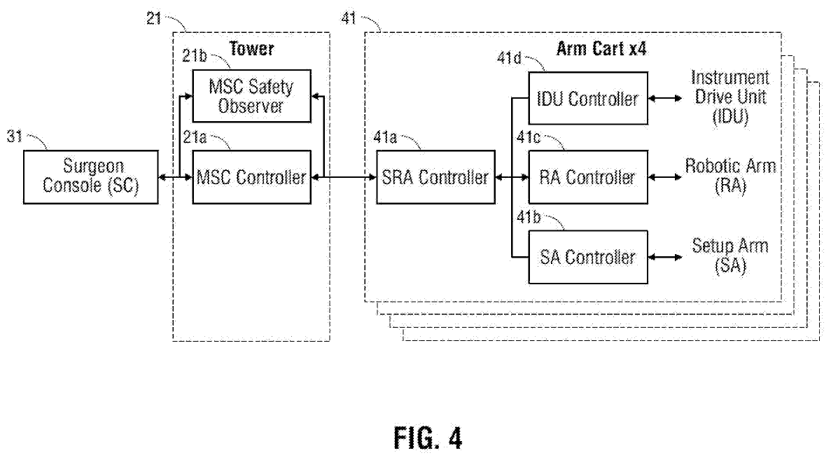

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40. The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62a and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference again to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 further includes a plurality of manual override buttons 53 disposed on the IDU 52 (see FIG. 1) and the setup arm 62, which may be used in a manual mode. For example, the clinician may press one of the buttons 53 to move the component associated with that button 53 (FIG. 1).

The joints 44a and 44b include an actuator 48a and 48b configured to drive the joints 44a, 44b, 44c relative to each other through a series of belts 45a and 45b or other mechanical linkages such as drive rods, cables, levers, and/or the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b such that the actuator 48b is configured to rotate each of the links 42b, 42c and the holder 46 relative to one another. More specifically, links 42b, 42c and the holder 46 are passively coupled to the actuator 48b which enforces rotation about a remote center point “P” that lies at an intersection of the first axis defined by the link 42a and the second axis defined by the holder 46. Thus, the actuator 48b controls the angle “0” between the first and second axes allowing for orientation of the surgical instrument 50. Due to the interlinking of the links 42a, 42b, 42c and the holder 46 via the belts 45a and 45b, the angles between the links 42a, 42b, 42c and the holder 46 are also adjusted in order to achieve the desired angle “θ.” In aspects, some or all of the joints 44a, 44b, 44c may include an actuator to obviate the need for mechanical linkages.

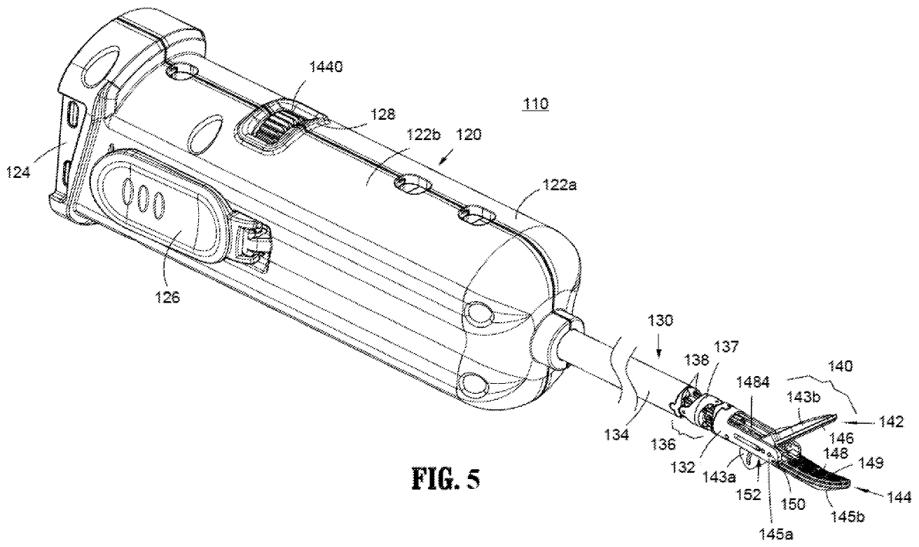

With reference to FIG. 4, in conjunction with FIGS. 1-3, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and/or other inputs. The controller 21a processes these input positions to determine desired drive commands for each joint of the robotic arm 40 and/or the IDU 52 and communicates these to the computer 41 of the robotic arm 40. The controller 21a also receives the actual joint angles and uses this information to determine force feedback commands that are transmitted back to the computer 31 of the surgical console 30 to provide haptic or other feedback through the handle controllers 38a and 38b. The handle controllers 38a and 38b include one or more haptic feedback vibratory devices that output haptic feedback although visual, audible, and/or other feedback is also contemplated. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an IDU controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a communicates the actual joint angles back to the controller 21a.

The setup arm controller 41b controls each of joints 63a and 63b and the rotatable base 64 of the setup arm 62 and calculates desired motor movement commands (e.g., motor torque) for the pitch axis. The setup arm controller 41b also controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

With respect to control of the robotic arm 40, initially, a pose of the handle controller controlling the robotic arm 40, e.g., the handle controller 38a, is transformed into a desired pose of the robotic arm 40 through a hand eye transform function executed by the controller 21a. The hand eye function is embodied in software executable by the controller 21a or any other suitable controller of the surgical robotic system 10. The pose of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limiting mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

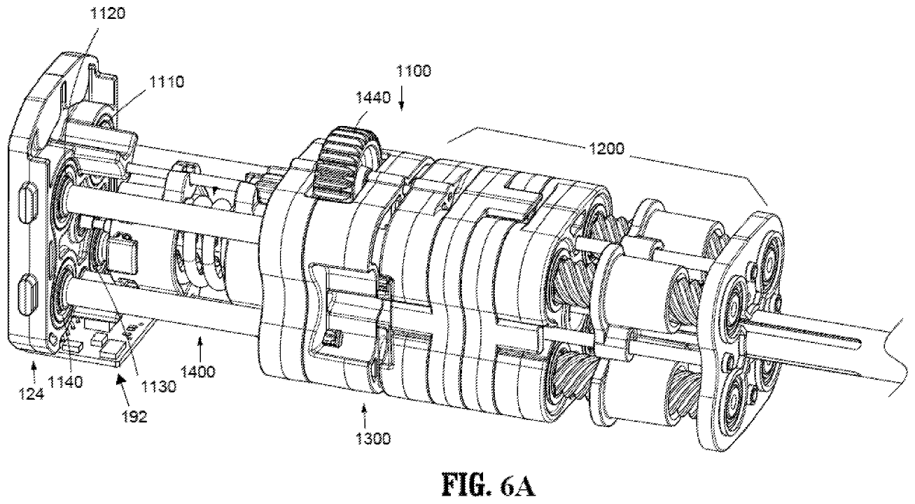

Turning to FIGS. 5-7, a surgical instrument 110 provided in accordance with this disclosure and configured for use with surgical robotic system 10 (FIG. 1) generally includes a housing 120, a shaft 130 extending distally from housing 120, an end effector assembly 140 extending distally from shaft 130, and an actuation assembly 1100 disposed within housing 120 and operably associated with end effector assembly 140. Instrument 110 is detailed herein as an articulating electrosurgical forceps configured for use with a surgical robotic system, e.g., surgical robotic system 10 (FIG. 1). However, the aspects and features of instrument 110 provided in accordance with this disclosure, detailed below, are equally applicable for use with other suitable surgical instruments, e.g., graspers, staplers, clip appliers, and/or in other suitable surgical systems, e.g., motorized, other power-driven systems, and/or manually-actuated surgical systems (including handheld instruments).

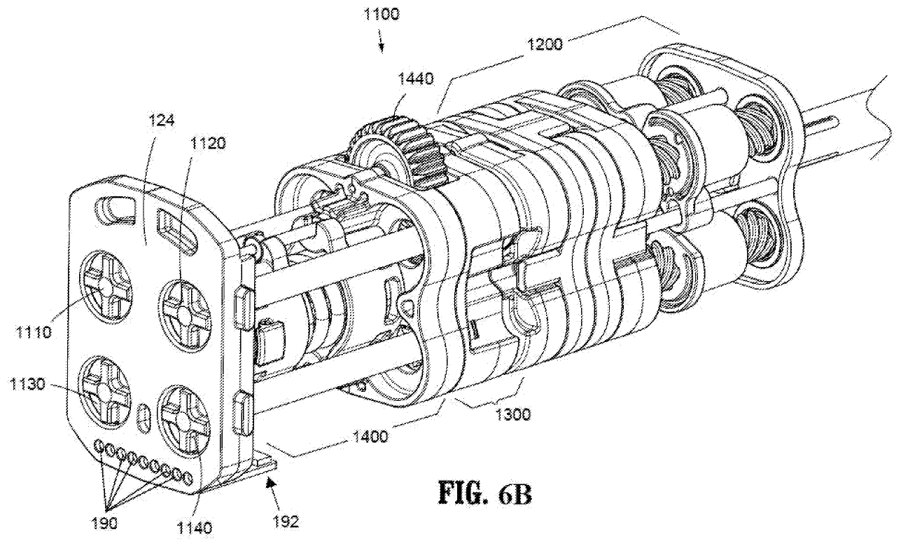

With particular reference to FIG. 5, housing 120 of instrument 110 includes first and second body portion 122a, 122b and a proximal face plate 124 that cooperate to enclose actuation assembly 1100 therein. Proximal face plate 124 includes through holes defined therein through which input couplers 1110-1140 (FIG. 6B) of actuation assembly 1100 extend. A pair of latch levers 126 (only one of which is illustrated in FIG. 5) extending outwardly from opposing sides of housing 120 enable releasable engagement of housing 120 with a robotic arm of a surgical robotic system, e.g., surgical robotic system 10 (FIG. 1). A window 128 defined through housing 120 permits thumbwheel 1440 to extend therethrough to enable manual manipulation of thumbwheel 1440 from the exterior of housing 120 to permit manual opening and closing of end effector assembly 140.

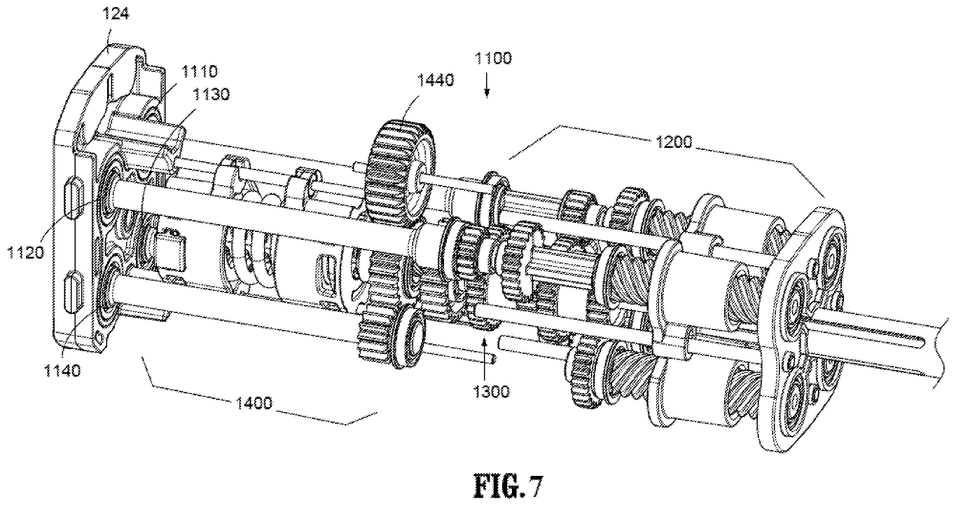

Referring also to FIGS. 6A-7, a plurality of electrical contacts 190 extend through one or more apertures defined through proximal face plate 124 to enable electrical communication between instrument 110 and surgical robotic system 10 (FIG. 1) when instrument 110 is engaged on a robotic arm thereof, e.g., for the communication of data, control, and/or power signals therebetween. As an alternative to electrical contacts 190 extending through proximal face plate 124, other suitable transmitter, receiver, and/or transceiver components to enable the communication of data, control, and/or power signals are also contemplated, e.g., using RFID, Bluetooth®, WiFi@, or via any other suitable wired, wireless, contacted, or contactless communication method. At least some of the electrical contacts 190 are electrically coupled with electronics 192 mounted on an interior side of proximal face plate 124, e.g., within housing 120. Electronics 192 may include, for example, a storage device, a communications device (including suitable input/output components), and a CPU including a memory and a processor. Electronics 192 may be mounted on a circuit board or otherwise configured, e.g., as a chip.

The storage device of electronics 192 stores information relating to surgical instrument such as, for example: the item number, e.g., SKU number; date of manufacture; manufacture location, e.g., location code; serial number; lot number; use information; setting information; adjustment information; calibration information; security information, e.g., encryption key(s), and/or other suitable additional or alternative data. The storage device of electronics 192 may be, for example, a magnetic disk, flash memory, optical disk, or other suitable data storage device.

As an alternative or in addition to storing the above noted information in the storage device of electronics 192, some or all of such information, e.g., the use information, calibration information, setting information, and/or adjustment information, may be stored in a storage device associated with surgical robotic system 10 (FIG. 1), a remote server, a cloud server, etc., and accessible via instrument 110 and/or surgical robotic system 10 (FIG. 1). In such configurations, the information may, for example, be updated by manufacturer provided updates, and/or may be applied to individual instruments, units of instruments (e.g., units from the same manufacturing location, manufacturing period, lot number, etc.), or across all instruments. Further still, even where the information is stored locally on each instrument, this information may be updated by manufacturer provided updates manually or automatically upon connection to the surgical robotic system 10 (FIG. 1).

Referring again to FIG. 5, shaft 130 of instrument 110 includes a distal segment 132 (which may at least partially define a clevis of end effector assembly 40), a proximal segment 134, and an articulating section 136 disposed between the distal and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal segment 132 of shaft 130 at the distal ends thereof and extend proximally from distal segment 132 of shaft 130, through articulating section 136 of shaft 130 and proximal segment 134 of shaft 130, and into housing 120, wherein articulation cables 138 operably couple with an articulation sub-assembly 1200 of actuation assembly 1100 (FIG. 6A) to enable selective articulation of distal segment 132 (and, thus end effector assembly 140) relative to proximal segment 134 and housing 120, e.g., about at least two axes of articulation (yaw and pitch articulation, for example). Articulation cables 138 are arranged in a generally rectangular configuration, although other suitable configurations are also contemplated. In some configurations, as an alternative, shaft 130 is substantially rigid, malleable, or flexible and not configured for active articulation. Articulation sub-assembly 1200 is described in greater detail below.

With respect to articulation of end effector assembly 140 relative to proximal segment 134 of shaft 130, actuation of articulation cables 138 may be accomplished in pairs. More specifically, in order to pitch end effector assembly 140, the upper pair of cables 138 are actuated in a similar manner while the lower pair of cables 138 are actuated in a similar manner relative to one another but an opposite manner relative to the upper pair of cables 138. With respect to yaw articulation, the right pair of cables 138 are actuated in a similar manner while the left pair of cables 138 are actuated in a similar manner relative to one another but an opposite manner relative to the right pair of cables 138. Other configurations of articulation cables 138 or other articulation actuators are also contemplated.

Continuing with reference to FIG. 5, end effector assembly 140 includes first and second jaw members 142, 144, respectively. Each jaw member 142, 144 includes a pair of proximal flags 143a, 145a and a distal body 143b, 145b, respectively, extending from the corresponding pair of proximal flags 143a, 145a. Distal bodies 143b, 145b define opposed tissue contacting surfaces 146, 148, respectively. Pairs of proximal flags 143a, 145a are pivotably coupled to one another about a pivot 150 (e.g., a pivot pin) and are operably coupled to one another via a cam assembly 152 including, as described in greater detail below, a cam pin slidably received within cam slots defined within the pairs of proximal flags 143a, 145a of jaw members 142, 144, respectively, to enable pivoting of jaw member 142 relative to jaw member 144 and distal segment 132 of shaft 130 between a spaced apart position (e.g., an open position of end effector assembly 140) and an approximated position (e.g., a closed position of end effector assembly 140) for grasping tissue between tissue contacting surfaces 146, 148. As an alternative to this unilateral configuration, a bilateral configuration may be provided whereby both jaw members 142, 144 are pivotable relative to one another and distal segment 132 of shaft 130.

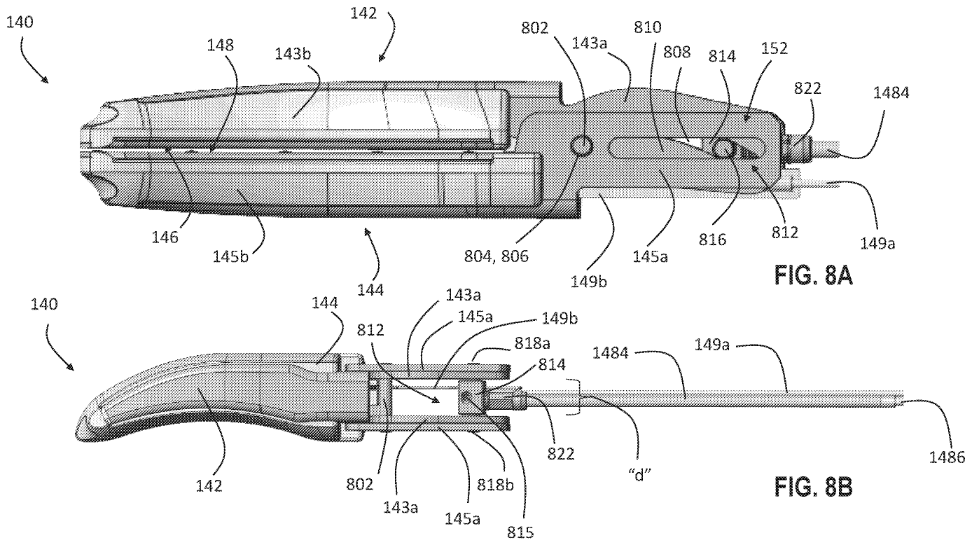

In aspects, a longitudinally extending knife channel 149 (only knife channel 149 of jaw member 144 is illustrated; the knife channel of jaw member 142 is similarly configured) is defined through the tissue contacting surface 146, 148 of one or both jaw members 142, 144. In such aspects, a knife assembly including a knife rod 149a (FIGS. 8A-8B) extending from housing 120 through shaft 130 to end effector assembly 140 and a knife blade 149b (FIGS. 8A-8B) disposed within end effector assembly 140 between jaw members 142, 144 is provided. The knife blade 149b (FIGS. 8A-8B) is selectively translatable through the knife channel(s) 149 and between the jaw member 142, 144 to cut tissue grasped between tissue contacting surfaces 146, 148 of jaw members 142, 144, respectively. The knife rod 149 (FIGS. 8A-8B) is operably coupled to a knife drive sub-assembly 1300 (FIG. 7) of actuation assembly 1100 (FIGS. 6A-6B) at a proximal end thereof to enable the selective actuation of the knife rod 149a (FIGS. 8A-8B) to, in turn, reciprocate the knife blade 149b (FIGS. 8A-8B) between jaw members 142, 144 to cut tissue grasped between tissue contacting surfaces 146, 148. As an alternative to a longitudinally advanceable mechanical knife, other suitable mechanical cutters are also contemplated, e.g., guillotine style cutters, as are energy based cutters, e.g., RF electrical cutters, ultrasonic cutters, etc., in static or dynamic configurations.

Referring still to FIG. 5, a drive tube 1484 is operably coupled to cam slot assembly 152 of end effector assembly 140, e.g., engaged with the cam pin thereof, such that longitudinal actuation of drive tube 1484 pivots jaw member 142 relative to jaw member 144 between the spaced apart and approximated positions. More specifically, urging drive tube 1484 proximally pivots jaw member 142 relative to jaw member 144 towards the approximated position while urging drive tube 1484 distally pivots jaw member 142 relative to jaw member 144 towards the spaced apart position. However, other suitable mechanisms and/or configurations for pivoting jaw member 142 relative to jaw member 144 between the spaced apart and approximated positions in response to selective actuation of drive tube 1484 are also contemplated. Drive tube 1484 extends proximally from end effector assembly 140 and is coupled to a drive rod 1486 that extends through shaft 130 and into housing 120 wherein drive rod 1486 is operably coupled with a jaw drive sub-assembly 1400 of actuation assembly 1100 (FIGS. 6A-6B) to enable selective actuation of end effector assembly 140 to grasp tissue therebetween and apply a jaw force within an appropriate jaw force range.

Tissue contacting surfaces 146, 148 of jaw members 142, 144, respectively, are at least partially formed from an electrically conductive material and are energizable to different potentials to enable the conduction of RF electrical energy through tissue grasped therebetween, although tissue contacting surfaces 146, 148 may alternatively be configured to supply any suitable energy, e.g., thermal, microwave, light, ultrasonic, ultrasound, etc., through tissue grasped therebetween for energy based tissue treatment. Instrument 110 defines a conductive conduit (not shown) through housing 120 and shaft 130 to end effector assembly 140 that may include lead wires, contacts, and/or electrically conductive components to enable electrical connection of tissue contacting surfaces 146, 148 of jaw members 142, 144, respectively, to an energy source (not shown), e.g., an electrosurgical generator, for supplying energy to tissue contacting surfaces 146, 148 to treat, e.g., seal, tissue grasped between tissue contacting surfaces 146, 148.

With additional reference to FIGS. 6A-7, as noted above, actuation assembly 1100 is disposed within housing 120 and includes an articulation sub-assembly 1200, a knife drive sub-assembly 1300, and a jaw drive sub-assembly 1400. Articulation sub-assembly 1200 is operably coupled between first and second input couplers 1110, 1120, respectively, of actuation assembly 1100 and articulation cables 138 (FIG. 5) such that, upon receipt of appropriate inputs into first and/or second input couplers 1110, 1120, articulation sub-assembly 1200 manipulates cables 138 (FIG. 5) to articulate end effector assembly 140 in a desired direction, e.g., to pitch and/or yaw end effector assembly 140. Articulation sub-assembly 1200 is described in greater detail below.

Knife drive sub-assembly 1300 is operably coupled between third input coupler 1130 of actuation assembly 1100 and the knife rod 149a (FIGS. 8A-8B) such that, upon receipt of appropriate input into third input coupler 1130, knife drive sub-assembly 1300 manipulates the knife rod 149a (FIGS. 8A-8B) to reciprocate the knife blade 149b (FIGS. 8A-8B) between jaw members 142, 144 to cut tissue grasped between tissue contacting surfaces 146, 148.

Jaw drive sub-assembly 1400 is operably coupled between fourth input coupler 1140 of actuation assembly 1100 and drive rod 1486 such that, upon receipt of appropriate input into fourth input coupler 1140, jaw drive sub-assembly 1400 pivots jaw members 142, 144 between the spaced apart and approximated positions to grasp tissue therebetween and apply a jaw force within an appropriate jaw force range.

Actuation assembly 1100 is configured to operably interface with a surgical robotic system, e.g., system 10 (FIG. 1), when instrument 110 is mounted on a robotic arm thereof, to enable robotic operation of actuation assembly 1100 to provide the above detailed functionality. That is, surgical robotic system 10 (FIG. 1) selectively provides inputs, e.g., rotational inputs to input couplers 1110-1140 of actuation assembly 1100 to articulate end effector assembly 140, grasp tissue between jaw members 142, 144, and/or cut tissue grasped between jaw members 142, 144. However, as noted above, it is also contemplated that actuation assembly 1100 be configured to interface with any other suitable surgical systems, e.g., a manual surgical handle, a powered surgical handle, etc.

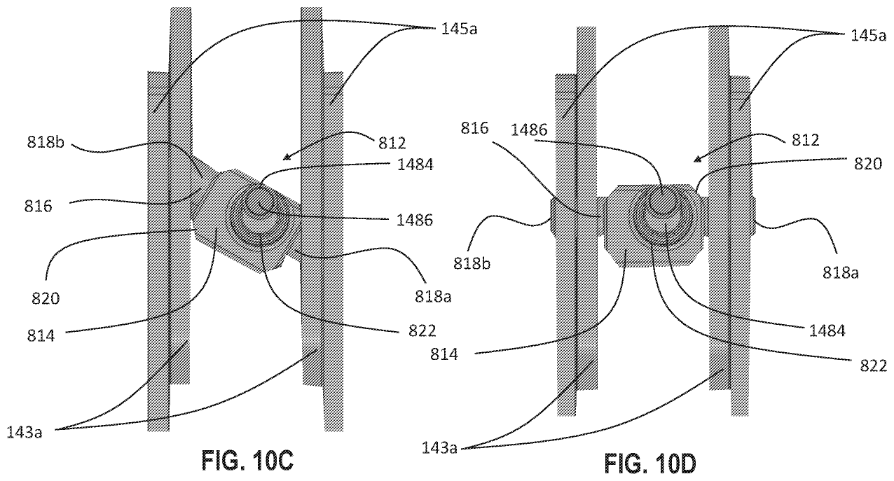

Turning to FIGS. 8A and 8B, as detailed above with reference to FIG. 5, first and second jaw members 142, 144, respectively, of end effector assembly 140 each includes a pair of proximal flags 143a, 145a and a distal body 143b, 145b, respectively, extending from the corresponding pair of proximal flags 143a, 145a. The proximal flags in each pair of proximal flags 143a, 145a are spaced apart from one another. Further, the pairs of proximal flags 143a, 145a may arranged to define a nested configuration, as shown, wherein the proximal flags of one of the jaw members, e.g., proximal flags 143a of jaw member 142, are disposed between, e.g., nested within, the proximal flags of the other jaw member, e.g., proximal flags 145a of jaw member 145. Alternatively, the pairs of proximal flags 143a, 145a may arranged in an offset configuration wherein one proximal flag of each pair of proximal flags 143a, 145a is disposed between the proximal flags of the other pair of proximal flags 143a, 145a and the other proximal flag of each pair of proximal flags 143a, 145a is disposed outside the proximal flags of the other pair of proximal flags 143a, 145a. In still other configurations, one of the jaw members 142, 144 includes only a single proximal flag disposed adjacent to one of the proximal flags of the pair of proximal flags of the other jaw member 142, 144. Additionally or alternatively, the proximal flag(s) associated with one of the jaw members 142, 144 may be attached to or formed with a clevis or other jaw support structure. Regardless of the particular arrangement of the proximal flags 143a, 145a, the inner-most proximal flags, e.g., proximal flags 143a (as shown), are transversely spaced apart from one another a distance “d” (see also FIGS. 10A-10D).

Continuing with reference to FIGS. 8A and 8B, as also detailed above with reference to FIG. 5, the pairs of proximal flags 143a, 145a are pivotably coupled to one another about a pivot 150 (FIG. 5) such as, for example, a pivot pin 802 extending though transversely-aligned pivot apertures 804, 806 defined through the pairs of proximal flags 143a, 145a. Pivot pin 802 may be welded in position, e.g., to one or both of proximal flags 145a, or may be retained in position in any other suitable manner such as, for example, via a clevis (not shown) or other suitable support structure disposed about proximal flags 145a to capture pivot pin 802 therein, as detailed below.

The pairs of proximal flags 143a, 145a are operably coupled to one another via a cam assembly 152 (FIG. 5). More specifically, each proximal flag of the pair of proximal flags 143a of jaw member 142 further defines a cam slot 808 of the cam assembly 152 (FIG. 5); likewise, each proximal flag of the pair of proximal flags 145a of jaw member 144 further defines a cam slot 810 of the cam assembly 152 (FIG. 5). The cam slots 810 of proximal flags 145a of jaw member 144 are aligned with one another and may extend in a linear, longitudinal orientation, while the cam slots 808 of proximal flags 143a of jaw member 142 are aligned with one another and may be curved (as shown) or angled relative to the cam slots 810 of proximal flags 145a of jaw member 144. In this manner, translation of a cam pin 816 through the cam slots 808, 810 urges jaw member 142 to pivot about pivot pin 802 and relative to jaw member 142, e.g., between the spaced apart and approximated positions.

Drive tube 1484 is operably coupled to cam slot assembly 152 (FIG. 5). More specifically, as noted above, drive tube 1484 is engaged with drive rod 1486 which, in turn, is operably coupled with jaw drive sub-assembly 1400 of actuation assembly 1100 (FIGS. 6A-6B) via a cam head assembly 812 to enable selective pivoting of jaw member 142 relative to jaw member 144, e.g., to grasp tissue therebetween. Drive tube 1484 and cam head assembly 812 (and, in some aspects, drive rod 1486) may be collectively referred to herein as the cam drive assembly.

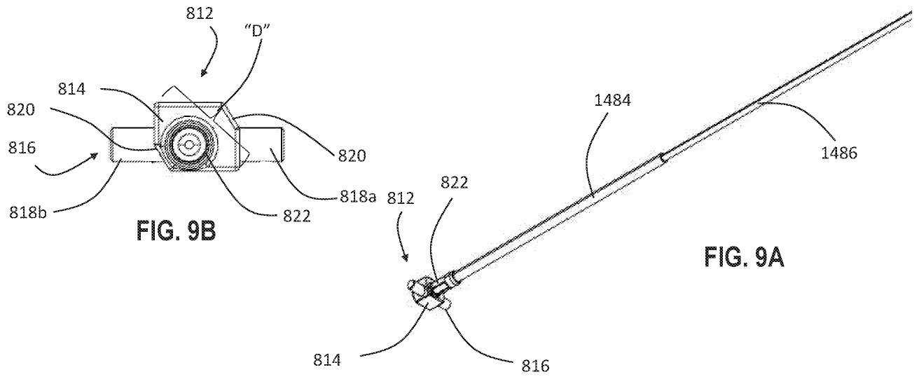

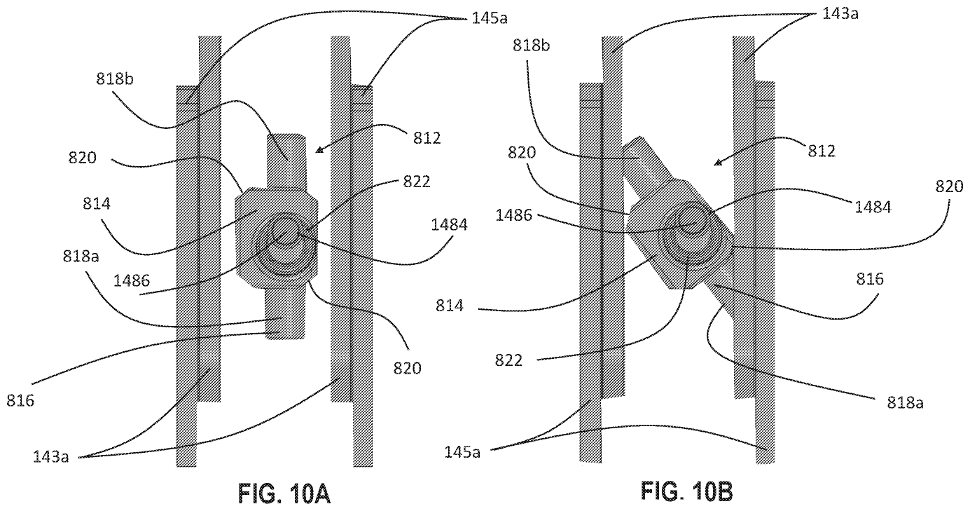

With additional reference to FIGS. 9A and 9B, cam head assembly 812 includes a cam block 814 and a cam pin 816 fixed relative to cam block 814 such that first and second cam pin portions 818a, 818b protrude transversely from opposite sides of cam block 814. Cam pin 816 may be fixed relative to cam block 814 via welding, via monolithically forming cam pin 816 and cam block 814 as a single component (in the same or multiple steps), or in any other suitable manner. Further, cam pin 816 need not be continuous; that is, cam pin 816 need not extend through (or entirely through) cam block 814 but, rather, may be formed from separate cam pin portions 818a, 818b protruding transversely from cam block 814. In aspects where cam pin 816 is continuous and welded to cam block 814, cam block 814 may include a weld aperture 815 extending therethrough and intersecting cam pin 816 to provide access to cam pin 816 within cam block 814 to facilitate welding of cam pin 816 to cam block 814. Weld aperture 815 may extend substantially perpendicularly to cam pin 816 and may be defined through a top surface of cam block 814, although other configurations are also contemplated.

Continuing with reference to FIGS. 8A-9B, cam block 814 defines diagonal dimensions “D” between opposing (actual or would-be) corners thereof. Each diagonal dimension “D,” in aspects, is greater than the distance “d” between proximal flags 143a of jaw member 142 such that, if cam block 814 defined a fully squared-off rectangle, with cam block 814 disposed between proximal flags 143a of jaw member 142 in a first orientation wherein cam pin 816 is substantially parallel to proximal flags 143a, rotation of cam block 814 to a second orientation wherein cam pin 816 is substantially perpendicular to proximal flags 143a would be inhibited or at least require contact between cam pin 816 and proximal flags 143a. As such, at least one corner of cam block 814 defines a chamfer 820, which may be an angled surface (as shown), rounded corner, or other suitable cut-out to reduce at least one diagonal dimension of cam block 814 to a distance that is less than distance “d,” thus enabling the above-noted rotation of cam block 814 from the first orientation to the second orientation. In aspects (as shown in FIG. 9B), a chamfer 820 is defined at either or both ends of one diagonal, while the other diagonal does not include chamfers. This configuration enables rotation of cam block 814 from the first orientation to the second orientation in only one direction, thereby preventing mis-installation of cam block 814 between jaw members 142, 144. Alternatively (as shown in FIGS. 10A-10D), a chamfer 820 may be defined at either or both ends of both diagonals. In aspects, cam block 814 defines at least one diagonal dimension “D” between opposing (actual or would-be) corners thereof that is less than the distance “d” between proximal flags 143a such that cam block 814 may be rotated from the first orientation to the second orientation without the need for chamfers.

Cam block 814 further includes a proximal extension 822 configured for at least partial receipt of drive tube 1484 to engage drive tube 1484 with cam block 814, e.g., via welding drive tube 1484 to proximal extension 822, crimping proximal extension 822 about a distal end portion of drive tube 1484, etc. Drive rod 1486 is engaged with drive tube 1484 and may extend partially through drive tube 1484, e.g., wherein drive rod 1486 is engaged within a proximal end portion of drive tube 1484, or may extend completely through drive tube 1484, e.g., wherein drive rod 1486 is engaged within a distal end portion of drive tube 1484 and/or to proximal extension 822 of cam block 814.

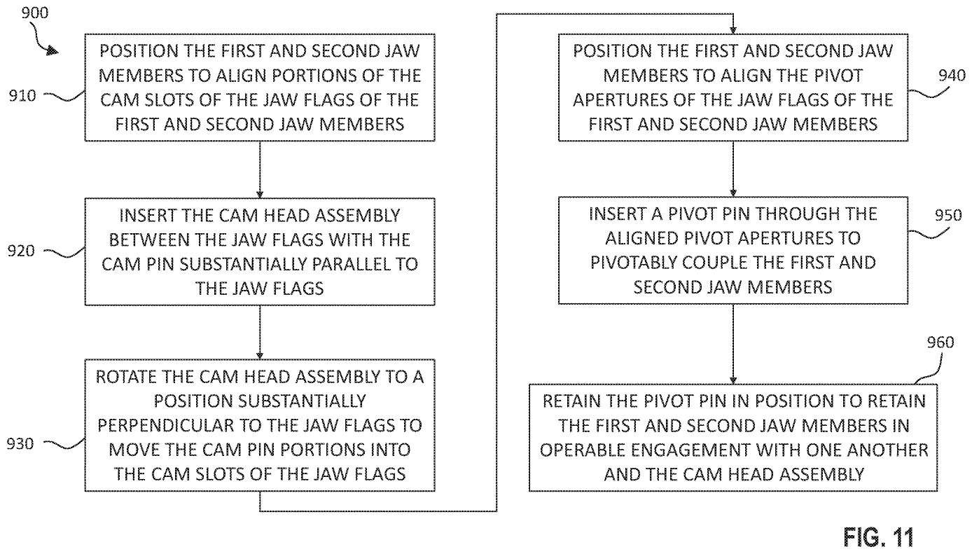

Turning to FIGS. 10A-10D and 11, in conjunction with FIGS. 8A and 8B, assembly of cam head assembly 812 with jaw members 142, 144 is detailed, e.g., in conjunction with method 900 (FIG. 11). Prior to assembly of cam head assembly 812 with jaw members 142, 144, cam pin 816 is engaged with cam block 814, e.g., via welding through weld aperture 815 (FIG. 8B). Drive tube 1484 is also engaged with proximal extension 822 of cam block 814 prior to assembly of cam head assembly 812 with jaw members 142, 144. Drive rod 1486 may also be engaged with dive tube 1484 prior to assembly of cam head assembly 812 with jaw members 142, 144, although drive tube 1484 and/or drive rod 1486 may alternatively be engaged after assembly of cam head assembly 812 with jaw members 142, 144.

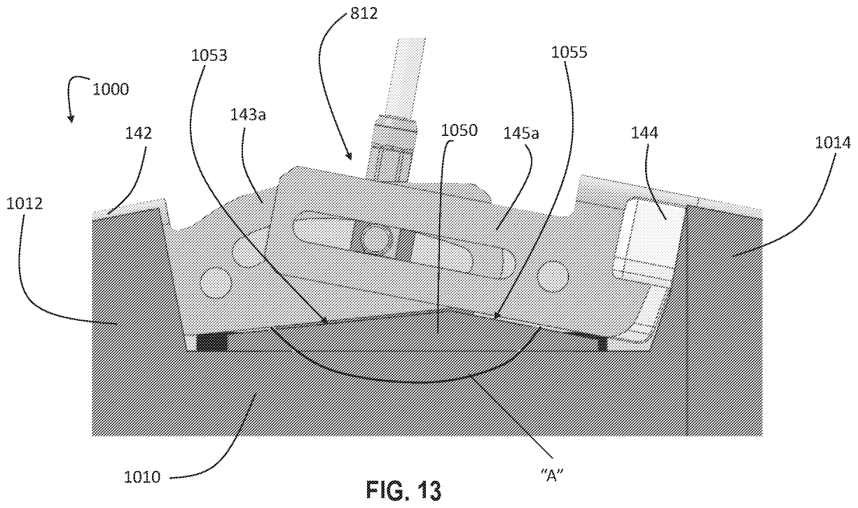

Initially, as indicated at 910, jaw members 142, 144 are positioned with proximal flags 143a disposed between proximal flags 145a and such that the cam slots 808, 810 (FIG. 8A) of the adjacent pairs of proximal flags 143a, 145a are partially aligned with one another thereby defining passages 811 (only one of which is shown, FIG. 13) extending through the pairs of cam slots 808, 810 (FIG. 8A). Passages 811 (FIG. 13) are sufficiently elongated to enable the rotation of cam pin 816 (when disposed between proximal flags 143a, 145a) into engagement with cam slots 808, 810 (FIG. 8A) despite cam pin 816 defining a length greater than the distance “d” between proximal flags 143a. Once the adjacent pairs of proximal flags 143a, 145a are partially aligned as detailed above, or prior thereto, cam head assembly 812 is inserted between proximal flags 143a of jaw member 142 in the first orientation wherein cam pin 816 is substantially parallel to proximal flags 143a, as shown in FIG. 10A and indicated at 920. Cam head assembly 812, more specifically, is positioned between proximal flags 143a such that cam pin 816 is disposed in substantial transverse alignment with the passages 811 (FIG. 13) defined through the partially aligned pairs of cam slots 808, 810 (FIG. 8A).

With cam head assembly 812 positioned as detailed above, cam head assembly 812 may then be rotated about a longitudinal axis thereof (e.g., a longitudinal axis extending substantially perpendicularly to cam pin 816), whereby chamfer(s) 820 enable rotation of cam head assembly 812, without contacting proximal flags 143a, from the first orientation to the second orientation wherein cam pin 816 is substantially perpendicular to proximal flags 143a. More specifically, as indicated at 930, and as illustrated progressively from FIGS. 10A to 10D, the rotation of cam head assembly 812 from the first orientation to the second orientation moves cam pin portions 818a, 818b through the passages 811 (FIG. 13) extending through the pairs of cam slots 808, 810 (FIG. 8A) and into final positions wherein cam pin portion 818a extends substantially perpendicularly through the cam slots 808, 810 (FIG. 8A) of jaw members 142, 144 on a first side of cam block 814 and wherein cam pin portion 818b extends substantially perpendicularly through the cam slots 808, 810 (FIG. 8A) of jaw members 142, 144 on a second, opposite side of cam block 814 (see FIG. 10D). Although cam block 814 is shown including chamfers 820 on both diagonals in FIGS. 10A-10D, it is also contemplated, as noted above, that cam block 814 includes chamfers 820 on only one diagonal to enable rotation of cam block 814 in only one direction, e.g., one-way rotation.

Referring back to FIGS. 8A and 8B, and with continued reference to FIG. 11, with assembly of cam head assembly 812 with jaw members 142, 144 complete as detailed above, assembly of end effector assembly 140 may continue. More specifically, as indicated at 940, with cam head assembly 812 operably coupled with jaw members 142, 144 as detailed above, jaw member 142 and/or jaw member 144 may be moved, e.g., rotated relative to one another to align pivot apertures 804, 806. This rotation of jaw member 142 and/or jaw member 144 moves cam slots 808, 810 relative to one another to inhibit disassembly of cam head assembly 812 from jaw members 142, 144, e.g., to inhibit disassembly in the reverse manner as the above-detailed assembly.

As indicated at 950, with pivot apertures 804, 806 of proximal flags 143a, 145a aligned with one another, pivot pin 802 may be inserted through the aligned pivot apertures 804, 806 to thereby pivotably couple jaw members 142, 144 with one another. Pivot pin 802 may thereafter be retained in position, as indicated at 960, thereby retaining jaw members 142, 144 in pivotable engagement with one another and retaining cam head assembly 812 in operable engagement with jaw members 142, 144. Pivot pin 802 may be retained in position via welding pivot pin 802 to either or both of proximal flags 145a (or proximal flags 143a). Alternatively, pivot pin 802 may be retained in “floating” engagement with proximal flags 143a, 145a, for example, via positioning a clevis (not explicitly shown) or other support structure at least partially about proximal flags 145a, thereby inhibiting withdrawal of pivot pin 802 from pivot apertures 804, 806 and maintaining jaw members 142, 144 in pivotable engagement without directly fixing pivot pin 802 to proximal flags 145a (or proximal flags 143a).

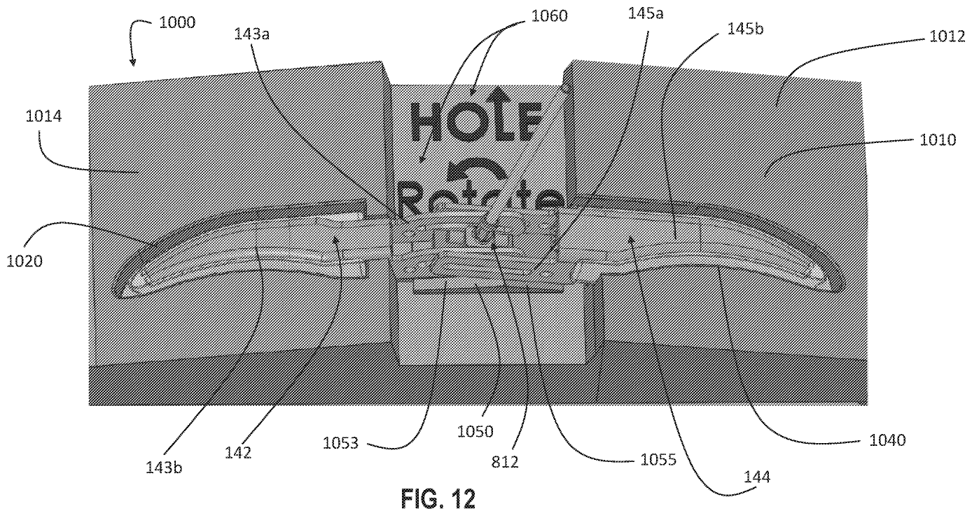

Turning to FIGS. 12 and 13, provided in accordance with aspects of this disclosure is a fixture 1000 configured to facilitate the above-detailed assembly of cam head assembly 812 with jaw members 142, 144. Fixture 1000, more specifically, includes a base 1010, a first jaw retainer 1020, a second jaw retainer 1040, and a support block 1050. First and second jaw retainers 1020, 1040 may be formed as depressions within respective first and second side portions 1012, 1014 of base 1010 on either side of support block 1050, or may be formed and/or positioned in any other suitable manner. The depressions or other suitable jaw retainers 1020, 1040 may be differentiated such that distal body 143b of jaw member 142 is only positionable within jaw retainer 1020 and/or such that distal body 145b of jaw member 144 is only positionable within jaw retainer 1040. First and second jaw retainers 1020, 1040 are configured to receive jaw member 142, 144 with tissue contacting surfaces 146, 148 (FIG. 8A) of jaw members 142, 144 facing downwardly towards base 1010, although other configurations are also contemplated.

Support block 1050, as noted above, is disposed between first and second side portions 1012, 1014 of base 1010. Support block 1050 includes first and second angled surfaces 1053, 1055 configured to support the pairs of proximal flags 143a, 145a, respectively, of respective first and second jaw members 142, 144. More specifically, second angled surfaces 1053, 1055 are oriented relative to one another such that, with distal bodies 143b, 145b of jaw members 142, 144 disposed within respective jaw retainers 1020, 1040 and proximal flags 143a, 145a of jaw members 142, 144 supported on respective angled surfaces 1053, 1055, cam slots 808, 810 of the adjacent pairs of proximal flags 143a, 145a are partially aligned with one another to define passages 811 which enable rotational passage of cam pin 816 therethrough, as detailed above. In aspects, angled surfaces 1053, 1055 may be disposed at an angle “A” relative to one another such that tissue contacting surfaces 146, 148 (FIG. 8A) of jaw members 142, 144 are disposed at angle “A” relative to one another of, in aspects, from about 150 degrees to about 175 degrees; in other aspects, from about 155 degrees to about 170 degrees; in still other aspects, from about 160 degrees to about 165 degrees; and in yet other aspects, of about 163 degrees.

Fixture 1000 may further include indicia 1060 indicating, for example, the proper insertion orientation of cam head assembly 812 (e.g., with reference to an orientation of weld aperture 815 (FIG. 8B)) and/or the proper direction of rotation of cam head assembly 812.

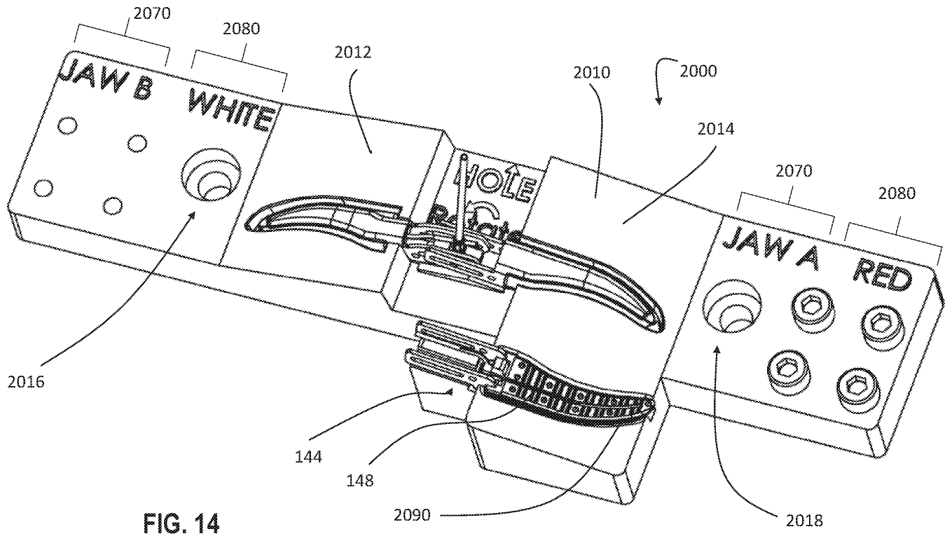

FIG. 14 illustrates another fixture 2000 provided in accordance with aspects of this disclosure and configured to facilitate the above-detailed assembly of cam head assembly 812 with jaw members 142, 144. Fixture 2000 may be configured similar to and include any or all of the features of fixture 1000 (FIGS. 12 and 13) as detailed above; thus, only difference between fixture 2000 and fixture 1000 (FIGS. 12 and 13) are described in detail below while similarities are summarily described or omitted entirely.

Fixture 2000 includes indicia 2070 on first and second side portions 2012, 2014 of base 2010 indicating which side portion 2012, 2014 corresponds to each jaw member 142, 144. Indicia 2080 may also be provided to indicate the wire cover color of the electrical lead wires that are configured to connect tissue contacting surfaces 146, 148 (FIG. 8A) of jaw members 142, 144, respectively, to the energy source (not shown), e.g., electrosurgical generator. For example, jaw member 142, which may be denoted as “jaw b,” may include a white cover on the lead wire thereof, while jaw member 144, which may be denoted as “jaw a,” may include a red cover on the lead wire thereof. Further, side portions 2012, 2014 of base 2010 of fixture 2000 may include wire retainers 2016, 2018, e.g., apertures (as shown), slots, tortuous pathways, recesses, channels, etc., configured to receive the jaw wires associated with respective jaw members 142, 144 to maintain the jaw wires out of the way during assembly.

Continuing with reference to FIG. 14, in aspects, fixture 2000 further includes an additional jaw retainer 2090 configured for use during other portions of assembly. For example, additional jaw retainer 2090 may be utilized to retain jaw member 144 therein (with tissue contacting surface 148 facing upwardly away from base 2010) to facilitate the operable coupling of the knife blade 149b (FIGS. 8A-8B) with jaw member 144.

It will be understood that various modifications may be made to the aspects and features disclosed herein. Therefore, the above description should not be construed as limiting, but merely as exemplifications of various configurations. Those skilled in the art will envision other modifications within the scope and spirit of the claims appended hereto.

What is claimed is:

1. A method of assembling an end effector assembly of a surgical instrument, the method comprising:
positioning a first proximal flag of a first jaw member between spaced apart second and third proximal flags of a second jaw member and adjacent to the second proximal flag such that a first cam slot defined through the first proximal flag is partially aligned with a second cam slot defined through the second proximal flag to define a first passage through the first and second cam slots;
inserting a cam head assembly between the first and third proximal flags in a first orientation wherein a first cam pin portion extending from a cam block of the cam head assembly is oriented substantially parallel relative to the first, second, and third proximal flags; and
rotating the cam head assembly from the first orientation to a second orientation, wherein the first cam pin portion is oriented substantially perpendicularly relative to the first, second, and third proximal flags, to thereby rotate the first cam pin portion through the first passage and into engagement within the first and second cam slots.

2. The method of assembly according to claim 1, wherein the first jaw member further includes a fourth proximal flag spaced apart from the first proximal flag, and wherein:
the positioning further includes positioning the fourth proximal flag adjacent to the third proximal flag such that a fourth cam slot defined through the fourth proximal flag is partially aligned with a third cam slot defined through the third proximal flag to define a second passage through the third and fourth cam slots;
the cam head assembly includes a second cam pin portion extending from the cam block opposite the first cam pin portion; and
the rotating rotates the second cam pin portion through the second passage and into engagement within the third and fourth cam slots.

3. The method of assembly according to claim 2, wherein positioning the fourth proximal flag adjacent to the third proximal flag includes positioning the fourth proximal flag on an interior side of the third proximal flag between the third proximal flag and the first proximal flag.

4. The method of assembly according to claim 2, wherein positioning the fourth proximal flag adjacent to the third proximal flag includes positioning the fourth proximal flag on an exterior side of the third proximal flag such that the third proximal flag is disposed between the fourth proximal flag and the first proximal flag.

5. The method of assembly according to claim 1, wherein the cam block includes a first corner defining a chamfer, and wherein the rotating includes rotating the first corner relative to the first proximal flag with a clearance therebetween defined by the chamfer.

6. The method of assembly according to claim 1, wherein the first jaw member includes a first jaw body extending distally from the first proximal flag and defining a first tissue contacting surface, wherein the second jaw member includes a second jaw body extending distally from the second and third proximal flags and defining a second tissue contacting surface, and wherein the positioning includes positioning the first and second tissue contacting surfaces relative to one another at an angle of from about 150 degrees to about 175 degrees.

7. The method of assembly according to claim 1, further comprising, after the rotating:
positioning the first proximal flag relative to the second and third proximal flags such that a first pivot aperture defined through the first proximal flag is aligned with second and third pivot apertures defined through the second and third proximal flags; and
inserting a pivot pin through the aligned first, second, and third pivot apertures to thereby pivotably couple the first and second jaw members with one another.

8. The method of assembly according to claim 7, further comprising retaining the pivot pin in engagement within the first, second, and third pivot apertures to thereby retain the first and second jaw members in pivotable engagement with one another.

9. The method of assembly according to claim 1, wherein the cam head assembly includes a drive tube engaged with and extending proximally from the cam block prior to the inserting.

10. The method of assembly according to claim 1, further comprising welding the first cam pin portion to the cam block prior to the inserting.

11. The method of assembly according to claim 1, wherein the positioning includes inserting the first and second jaw members into a fixture, and wherein the inserting and the rotating are performed with the first and second jaw members disposed within the fixture.

12. An end effector assembly of a surgical instrument, the end effector assembly comprising:
a first jaw member including a first pair of proximal flags and a distal body extending distally from the first pair of proximal flags and defining a first tissue contacting surface, the proximal flags of the first pair of proximal flags defining a transverse distance therebetween;
a second jaw member including a second pair of proximal flags and a distal body extending distally from the second pair of proximal flags and defining a second tissue contacting surface, wherein the first pair of proximal flags is disposed between the proximal flags of the second pair of proximal flags;
a pivot pivotably coupling the first and second pairs of proximal flags with one another to enable pivoting of at least one of the first or second tissue contacting surfaces relative to another of the first or second tissue contacting surfaces between spaced apart and approximated positions for grasping tissue therebetween; and
a cam drive assembly including a cam block and a cam pin fixed relative to the cam block, the cam block disposed between the proximal flags of the first pair of proximal flags with the cam pin operably engaging cam slots defined within the proximal flags of the first and second pairs of proximal flags,
wherein the cam block has a rectangular configuration defining a first diagonal transverse dimension and a second diagonal transverse dimension opposite the first diagonal transverse dimension, the first diagonal transverse dimension greater than the transverse distance and the second diagonal transverse dimension less than the transverse distance.

13. The end effector assembly according to claim 12, wherein the first diagonal transverse dimension extends between first and second corners of the cam block and wherein the second diagonal transverse dimension extends between first and second chamfers defined in the cam block.

14. The end effector assembly according to claim 12, wherein the cam pin extends through the cam block, wherein the cam block defines an aperture extending perpendicularly relative to the cam pin, and wherein the cam pin is welded to the cam block within the aperture.

15. The end effector assembly according to claim 12, wherein the cam drive assembly further includes a proximal extension configured to engage a drive tube with the cam block.

16. An end effector assembly of a surgical instrument, the end effector assembly comprising:
a first jaw member including a pair of first proximal flags and a distal body extending distally from the pair of first proximal flags and defining a first tissue contacting surface;
a second jaw member including at least one second proximal flag and a distal body extending distally from the at least one second proximal flag and defining a second tissue contacting surface;
a pivot pivotably coupling the pair of first proximal flags and the at least one second proximal flag with one another to enable pivoting of at least one of the first or second tissue contacting surfaces relative to another of the first or second tissue contacting surfaces between spaced apart and approximated positions for grasping tissue therebetween; and
a cam drive assembly including a cam block and a cam pin fixed relative to the cam block, the cam block disposed between the first proximal flags of the pair of first proximal flags with the cam pin operably engaging cam slots defined within the first proximal flags of the pair of first proximal flags, wherein the cam block includes a first diagonal defined between a pair of diagonally opposed chamfered corners.

17. The end effector assembly according to claim 16, wherein the first proximal flags of the pair of first proximal flags define a transverse distance therebetween, and wherein the first diagonal defines a diagonal transverse distance that is less than the transverse distance.

18. The end effector assembly according to claim 16, wherein the cam block further includes a second diagonal opposite the first diagonal and defined between a pair of diagonally opposed unchamfered corners.

19. The end effector assembly according to claim 16, wherein the at least one second proximal flag defines a cam slot, and wherein the cam pin is operably engaged within the cam slot of the at least one second proximal flag.

20. The end effector assembly according to claim 19, wherein the at least one second proximal flag includes a pair of second proximal flags, wherein the pair of first proximal flags is disposed between the second proximal flags of the pair of second proximal flags.
